(19) United States
(12) Patent Application Publication
(10) Pub. No.: US 2026/0263035 A1
(43) Pub. Date: Sep. 10, 2026

(54) BODY CAVITY INSERTABLE ULTRASOUND DEVICE INTEGRALLY EQUIPPED WITH AN ULTRASOUND TRANSDUCER FOR IMAGING, AND AN ULTRASOUND TREATMENT APPARATUS INCLUDING THE SAME

(71) Applicant: KORUST CO., LTD., Anyang-SI, Gyeonggi-do (KR)

(72) Inventor: Sung-Chan CHO, Seongnam-si (KR)

(73) Assignee: KORUST CO., LTD., Anyang-SI, Gyeonggi-do (KR)

(21) Appl. No.: 19/153,673

(22) PCT Filed: Nov. 20, 2023
(86) PCT No.: PCT/KR2023/018637

§ 371 Date: Aug. 5, 2025

(30) Foreign Application Priority Data
Feb. 8, 2023 (KR) 10-2023-0016652

(51) Int. Cl. A61B 8/12; A61B 8/00

(52) U.S. Cl. CPC A61B 8/12; A61B 8/445; A61B 8/4494; A61B 8/461; A61B 8/54

(57) ABSTRACT

The body cavity insertable ultrasound device includes a support bar extending in a longitudinal direction; an ultrasound probe comprising one or more ultrasound transducers supported by the support bar and configured to be inserted into a body cavity and to emit focused ultrasound; an imaging ultrasound transducer configured to acquire an image of tissue on which the focused ultrasound emitted from the ultrasound transducer acts; and a sealing cover configured to surround at least a portion of the ultrasound probe and the support bar.

### TECHNICAL FIELD

The present invention relates to a body cavity insertable ultrasound device configured to be inserted into a body cavity such as the nasal cavity, oral cavity, pharynx, vagina, or urethra to perform an ultrasound procedure.

### BACKGROUND ART

Ultrasound has been used for various treatments and procedures, and for example, a method has been introduced in which ultrasound is non-invasively applied within a body cavity such as the oral cavity to treat diseases using heat. As another example, a method has been introduced in which a body cavity insertable ultrasound device is inserted into the urethra to irradiate focused ultrasound to the prostate, thereby reducing tissue and treating benign prostatic hyperplasia.

Benign prostatic hyperplasia is a very common disease in elderly men. Although there are treatment methods to alleviate the condition using medication, such methods often have limited effectiveness or require cumbersome procedures. Surgical removal of the prostate is another treatment option, but it poses the risk of serious side effects. Recently, a method involving the insertion of a heat generator into the urethra to apply heat to the prostate has also been used. However, this method requires MRI for positioning, and instead of focusing heat above 60° C. on a specific region, it broadly applies heat to the prostate. As a result, depending on the patient's condition, the accuracy may be reduced, unwanted areas may be exposed to heat, requiring a separate cooling device, and the equipment itself is extremely expensive.
Prior Art Document: U.S. Patent Application Publication No. US2008/0027423

### DETAILED DESCRIPTION OF THE INVENTION

### Technical Problem

The problem to be solved by the present invention is to provide a body cavity insertable ultrasound device capable of performing efficient ultrasound treatment while minimizing the load applied to the body cavity tissue.

### Technical Solutions

A body cavity insertable ultrasound device according to an embodiment of the present invention includes a support bar extending in a longitudinal direction; an ultrasound probe including one or more ultrasound transducers supported by the support bar and configured to be inserted into a body cavity and to emit focused ultrasound; an imaging ultrasound transducer configured to acquire an image of tissue on which the focused ultrasound emitted from the ultrasound transducer acts; and a sealing cover configured to surround at least a portion of the ultrasound probe and the support bar.

The ultrasound probe may be configured to be capable of linear movement.

The imaging ultrasound transducer may include a piezoelectric element for A-mode ultrasound imaging, and the piezoelectric element for A-mode ultrasound imaging may be installed on the ultrasound probe so as to move linearly together with the ultrasound probe.

The imaging ultrasound transducer may include a piezoelectric element for B-mode ultrasound imaging.

The ultrasound probe may be configured to be capable of linear movement, and the piezoelectric element for B-mode ultrasound imaging may be configured to extend along the direction of the linear movement of the ultrasound probe.

The ultrasound probe may include a plurality of ultrasound transducers having different focal depths, and the plurality of ultrasound transducers may be arranged in order of their focal depths.

The ultrasound probe may be configured to be capable of linear movement, and may be configured to emit the focused ultrasound while passing through a desired treatment region by the linear movement. The ultrasound probe may be further configured to move in a direction in which the ultrasound transducer having the greatest focal depth among the plurality of ultrasound transducers enters the treatment region first during the emission of the focused ultrasound.

The plurality of ultrasound transducers having different focal depths may be individually controlled such that, when the focused ultrasound is emitted while passing through the treatment region, the focused ultrasound is not emitted if its focal point falls outside the treatment region.

According to an embodiment of the present invention, a body cavity insertable ultrasound device includes: an outer case; a body rotatably disposed within the outer case about a predetermined rotation axis; a support bar fastened to the body so as to be movable in a longitudinal direction relative to the body and rotatable together with the body about the rotation axis; an ultrasound probe including one or more ultrasound transducers supported by the support bar and configured to be inserted into a body cavity and to emit focused ultrasound; an imaging ultrasound transducer configured to acquire an image of tissue on which the focused ultrasound emitted from the ultrasound transducers acts; and a sealing cover fastened to the body so as to rotate together with the body about the rotation axis and configured to surround and at least a portion of the ultrasound probe the support bar.

According to an embodiment of the present invention, an ultrasound treatment apparatus includes: a support bar extending in a longitudinal direction; an ultrasound probe supported by the support bar, the ultrasound probe including one or more ultrasound transducers configured to be inserted into a body cavity and to emit focused ultrasound and configured to be capable of linear movement; an imaging ultrasound transducer configured to acquire an image of tissue on which the focused ultrasound emitted from the ultrasound transducers acts; a sealing cover configured to surround at least a portion of the ultrasound probe and the support bar; a display device configured to display the image acquired by the imaging ultrasound transducer; and a power/control device configured to control the linear movement of the ultrasound probe and the operation of the one or more ultrasound transducers.

The display device may be configured to allow a treatment region to be indicated on the acquired image, and the power/control device may be configured to control the movement of the ultrasound probe and the operation of the one or more ultrasound transducers in consideration of the treatment region.

The one or more ultrasound transducers may be configured to respectively generate focused ultrasound beams having different focal depths. The power/control device may be configured to control linear movement of the ultrasound probe so that the ultrasound probe passes through the treatment region. In addition, the power/control device may be configured to individually control the plurality of ultrasound transducers having different focal depths such that, when the ultrasound probe passes through the treatment region, focused ultrasound is not emitted if the focal point of the focused ultrasound falls outside the treatment region.

The power/control device may be configured to control the ultrasound probe to emit focused ultrasound while passing through a desired treatment region by linear movement, and to control the ultrasound probe to move in a direction in which the ultrasound transducer having the greatest focal depth among the plurality of ultrasound transducers enters the treatment region first.

The imaging ultrasound transducer may include a piezoelectric element for A-mode ultrasound imaging, and the piezoelectric element for A-mode ultrasound imaging may be installed on the ultrasound probe so as to move linearly together with the ultrasound probe.

The imaging ultrasound transducer may include a piezoelectric element for B-mode ultrasound imaging, and the piezoelectric element for B-mode ultrasound imaging may be configured to extend along the direction of linear movement of the ultrasound probe.

The ultrasound probe may be further configured to be rotatable about a predetermined rotation axis in addition to being capable of linear movement.

### Effect of the Invention

According to the present invention, effective ultrasound irradiation can be achieved while minimizing the load applied to a body cavity such as the urethra, by controlling the movement of the ultrasound probe and the operation of the ultrasound transducer that generates focused ultrasound without moving the sealing cover. In particular, by integrally providing an ultrasound transducer for image acquisition, a treatment region can be displayed on a cross-sectional image of the target tissue, and based on this, the movement of the ultrasound probe and the operation of the focused ultrasound transducer can be controlled.

### BRIEF DESCRIPTION OF THE DRAWINGS

FIG. 1 is a perspective view of a body cavity insertable ultrasound device according to an embodiment of the present invention.

FIG. 2 is a partially exploded perspective view of the body cavity insertable ultrasound device according to an embodiment of the present invention.

FIG. 3 is a cross-sectional view of the body cavity insertable ultrasound device according to an embodiment of the present invention.

FIG. 4 is an enlarged perspective view of an ultrasound probe installed in a sealing cover of the body cavity insertable ultrasound device according to an embodiment of the present invention.

FIG. 5 is a plan view of the ultrasound probe installed in the sealing cover of the body cavity insertable ultrasound device according to an embodiment of the present invention.

FIG. 6 is a diagram illustrating a process of performing an ultrasound procedure using the body cavity insertable ultrasound device according to an embodiment of the present invention.

FIG. 7 is a drawing showing an ultrasound probe and an ultrasound transducer for imaging in a body cavity insertable ultrasound device according to another embodiment of the present invention.

FIG. 8 is an exploded perspective view of FIG. 7.

FIG. 9 is a perspective view of a body cavity insertable ultrasound device according to another embodiment of the present invention.

FIG. 10 is a view showing the body cavity insertable ultrasound device of FIG. 9 with a portion of an external case receiving the device removed.

### BEST MODE FOR CARRYING OUT THE INVENTION

Hereinafter, embodiments of the present invention will be described in detail with reference to the accompanying drawings. The following detailed description of the embodiments is provided to enable those skilled in the art to readily carry out the invention, with reference to the accompanying drawings. However, the present invention may be embodied in various different forms and is not limited to the embodiments described herein.

In describing the components of the present invention, terms such as first, second, A, B, (a), (b), and the like may be used. These terms are merely used to distinguish one component from another, and do not imply any limitation on the nature, sequence, or order of the components. When a component is described as being “connected,” “coupled,” or “joined” to another component, it should be understood that the component may be directly connected, coupled, or joined to the other component, or that one or more other components may be interposed therebetween.

The body cavity insertable ultrasound device according to an embodiment of the present invention is configured to perform ultrasound procedures, such as inducing thermal lesions within tissue by ultrasound energy, by irradiating ultrasound while being inserted into a body cavity such as the oral cavity, nasal cavity, pharynx, vagina, or urethra. In particular, the body cavity insertable ultrasound device according to an embodiment of the present invention may be used as a device that is inserted into the urethra to irradiate focused ultrasound to the prostate.

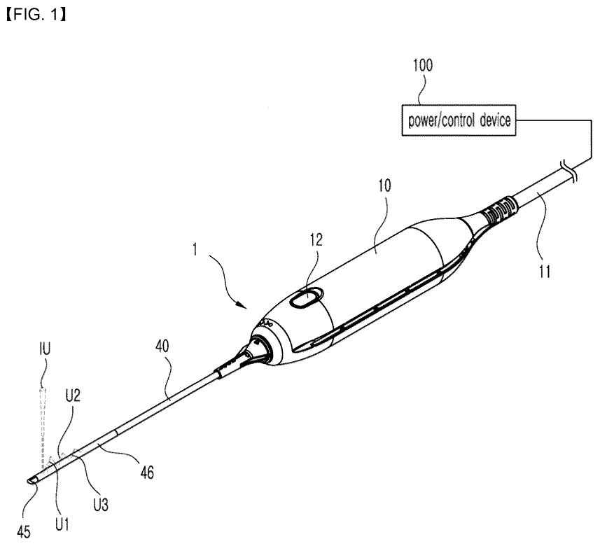

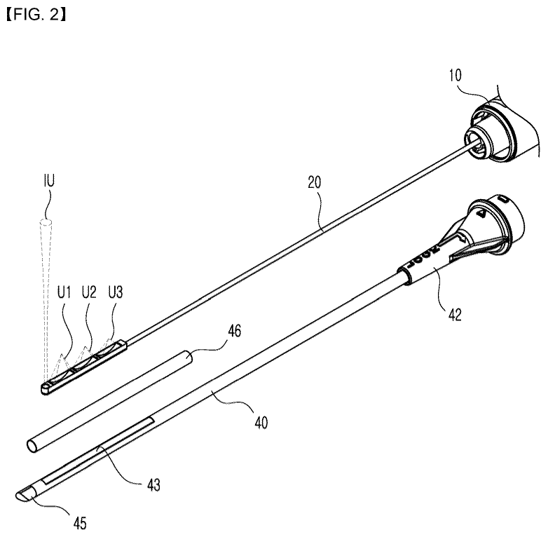

Referring to FIGS. 1 and 2, a body cavity insertable ultrasound device 1 according to an embodiment of the present invention includes a handle 10, a support bar 20, an ultrasound probe 30, and a sealing cover 40. The ultrasound probe 30, which is supported by the support bar 20, may be inserted into a body cavity, and to this end, at least a portion, i.e., the distal end of the ultrasound probe 30 and the support bar 20 may be formed to have a size and shape suitable for insertion into a body cavity such as the urethra.

The handle 10 is formed to be grasped by a practitioner performing the ultrasound procedure. As illustrated in FIG. 1 by way of example, the handle 10 may be configured to connect to an external power/control device 100 via a power cable 11, and may include an on/off button 12 for controlling the generation of ultrasound based on the power state. The power/control device 100 may be configured to supply pulsed power for ultrasound generation and to perform various controls for the ultrasound procedure. The controller for controlling the operation of the ultrasound probe 30 may be implemented as the power/control device 100.

As shown in FIG. 2, the support bar 20 extends from the distal end of the handle 10 and may have a rod-like shape. Since the distal end of the support bar 20 may be inserted into a body cavity during an ultrasound procedure, the support bar 20 may be formed as a rod having a relatively small diameter suitable for insertion into the body cavity. Meanwhile, in another embodiment of the present invention, the support bar 20 may be connected to a separate support device instead of the handle.

The support bar 20 extending from the handle 10 is sealed by the sealing cover 40, and the interior of the support bar 20 may be filled with an ultrasound transmission medium so that ultrasound generated by the ultrasound probe 30, which is disposed within the sealing cover 40, can be transmitted. The sealing cover 40 surrounds the support bar 20 and the ultrasound probe 30 supported thereby, and serves to seal the ultrasound transmission medium for ultrasound propagation. The ultrasound transmission medium not only acts as a medium for transmitting ultrasound, but also performs a cooling function by absorbing heat generated during the operation of the ultrasound probe 30. The structure for supplying and circulating the ultrasound transmission medium will be described below.

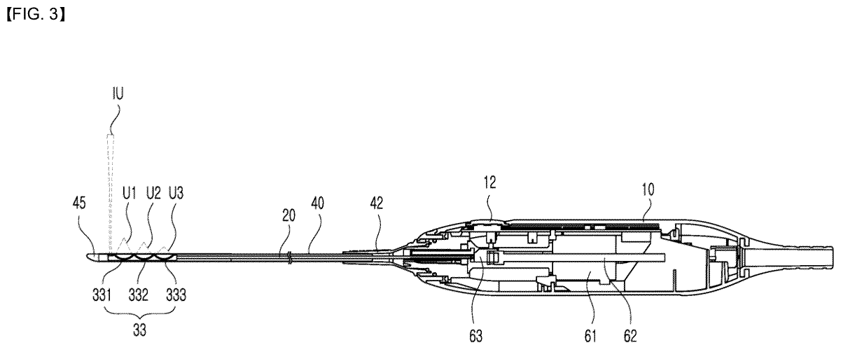

The ultrasound probe 30 generates ultrasonic vibrations upon application of pulsed power. Referring to FIGS. 2 and 3, the ultrasound probe 30 includes an ultrasound transducer housing 31 and an ultrasound transducer 33. The ultrasound transducer 33 may be composed of a piezoelectric material layer, such as piezoelectric ceramic, and a pair of electrodes formed on both sides of the material layer, as is conventionally known. When pulsed power is applied to both electrodes, the piezoelectric layer is configured to generate ultrasonic vibrations. Although not shown in the drawings, a power line for applying pulsed power to the ultrasound transducer 33 may be provided. For example, the power line may pass through the handle 10 and the support bar 20 and be electrically connected to the electrodes on both sides of the ultrasound transducer 33. For instance, the support bar 20 may have a hollow structure with a through-hole, and the power line may pass through this through-hole to connect to the ultrasound transducer 33. The ultrasound transducer 33 may include one or more ultrasound transducers 331, 332, 333 arranged along the longitudinal direction of the support bar 20. While the drawings illustrate an example including three ultrasound transducers 331, 332, and 333, a single ultrasound transducer may be provided, or two, four, or more ultrasound transducers may also be used.

The ultrasound transducers 331, 332, and 333 may be configured to focus ultrasound waves U1, U2, and U3 at desired positions. The ultrasound transducers 331, 332, and 333 may be configured to have different focal lengths, i.e., different focal depths. For example, the ultrasound transducers 331, 332, and 333 may be formed in various shapes capable of generating focused ultrasound, such as a concave curved surface, a concave cylindrical surface, a spherical surface, or a partially truncated spherical surface.

The ultrasound probe 30 is supported by the support bar 20. For example, the housing 31 of the ultrasound probe 30 may be fixed to the distal end of the support bar 20, thereby allowing the ultrasound probe 30 to be supported by the support bar 20.

The sealing cover 40 that houses the ultrasound probe 30 is provided. The sealing cover 40 may be fastened to the handle 10 while surrounding the support bar 20 and the ultrasound probe 30. Referring to FIGS. 1 and 2, the sealing cover 40 may have a hollow pipe shape with an elongated rod-like form, similar to the support bar 20. For example, the sealing cover 40 may include a tube member 41, a fastening member 42 that is fastened to one end of the tube member 41 and configured to be coupled to the handle 10, and an end cap 45 that is fastened to the distal end of the tube member 41. The end cap 45, located at the front end, is preferably formed of a soft material such as silicone or rubber, with a pointed tip to facilitate insertion into the urethra.

The tube member 41 is formed to accommodate the support bar 20 and the ultrasound probe 30. The tube member 41 may include an ultrasound transmission window 43 through which ultrasound generated by the ultrasound probe 30 can pass. For example, the tube member 41 may be formed of a metal material such as stainless steel or a plastic material. Although not shown in the drawings, a sealing film 46 capable of fluidly sealing the ultrasound transmission window 43 may be applied to the tube member 41. The sealing film 46 may be made of a material that seals the ultrasound transmission medium filled in the front space of the ultrasound probe 30 while allowing the ultrasound generated by the ultrasound probe 30 to pass through.

According to an embodiment of the present invention, the ultrasound probe 30 is configured to be capable of linear movement. By configuring the support bar 20 to be axially movable, the ultrasound probe 30 is also configured to move linearly together with the support bar 20. The support bar 20 is fastened to the handle 10 in a manner that allows axial movement. For example, a motor 61, such as a step motor, arranged within the handle 10 may provide driving force to cause longitudinal displacement of the support bar 20. Referring to FIG. 3, the support bar 20 may be screw-coupled to a rotating element 63 connected to an output shaft 62 of the motor 61, in a manner that restricts the rotation of the support bar 20, such that linear motion is produced in response to the rotation of the rotating element 63. When the rotating element 63 rotates by the operation of the motor 61, the support bar 20, which is screw-coupled to the rotating element 63, may perform linear movement along its axis. In this case, the ultrasound transmission window 43 of the sealing cover 40 may be formed longer than the ultrasound probe 30 so as to accommodate the linear movement of the ultrasound probe 30. Through the linear movement mechanism of the ultrasound probe 30, the irradiation position of ultrasound can be changed without moving the handle 10, thereby enabling the formation of a linear thermal lesion.

The space inside the sealing cover 40, in which the ultrasound probe 30 is disposed, is configured to be filled with water, which serves as the ultrasound transmission medium, and to allow the circulated flow of the filled water. The supplied water fills the front of the ultrasound transducer 33, thereby enabling the propagation of ultrasound. According to an embodiment of the present invention, a water supply tube 51, which serves as an ultrasound transmission medium supply tube, is provided to supply water to the space where the ultrasound probe 30 is disposed. The water supply tube 51 may extend from the handle 10 to the ultrasound probe 30. For example, the water supply tube 51 may extend within the sealing cover 40 in parallel with the support bar 20.

The rear end of the water supply tube 51, which is fastened to the handle 10, may be fluidly connected to a water supply pipe 81 that supplies water from an external source. Through this connection, water pumped by a water pump (not shown) can be introduced into the water supply tube 51 via the water supply pipe. The water introduced through the water supply tube 51 may be configured to fill the space around the ultrasound probe 30 and the internal space of the sealing cover 40, and then be discharged. For example, the inner space of the sealing cover 40 may be fluidly connected to a drainage pipe, so that the water supplied through the water supply tube 51 fills the internal space of the sealing cover 40 and is then discharged through the drainage pipe. In this manner, a circulation structure may be implemented in which water, serving as the ultrasound transmission medium, fills and is discharged from the space inside the sealing cover 40 where the ultrasound probe 30 is disposed. This enables both ultrasound propagation and a cooling function.

As described above, the ultrasound probe 30 is configured to be capable of linear movement, and the support bar 20 and the ultrasound probe 30 may be configured to move together within the sealing cover 40 by the operation of the motor 61. During this movement, the housing 31 of the ultrasound probe 30 is configured to move relative to the water supply tube 51. In other words, while the support bar 20 and the ultrasound probe 30 move inside the sealing cover 40, the water supply tube 51 remains stationary. To prevent the housing 31 of the ultrasound probe 30 from detaching from the water supply tube 51, a fastening member, such as a fastening tape 35, may secure the housing 31 and the water supply tube 51 together. Here, the fastening tape 35 is fixed to the housing 31 while allowing relative movement with respect to the water supply tube 51. Through this structure, the ultrasound probe 30 can move linearly along the longitudinal direction of the support bar 20.

Meanwhile, the body cavity insertable ultrasound device 1 according to an embodiment of the present invention may include an imaging ultrasound transducer 34 for acquiring an ultrasound image of the target tissue. The imaging ultrasound transducer 34 may be a general ultrasound transducer used for diagnostic purposes. The imaging ultrasound transducer 34 emits ultrasound IU for imaging and acquires an ultrasound image based on the reflected signals. A description of other components required for acquiring ultrasound images is omitted.

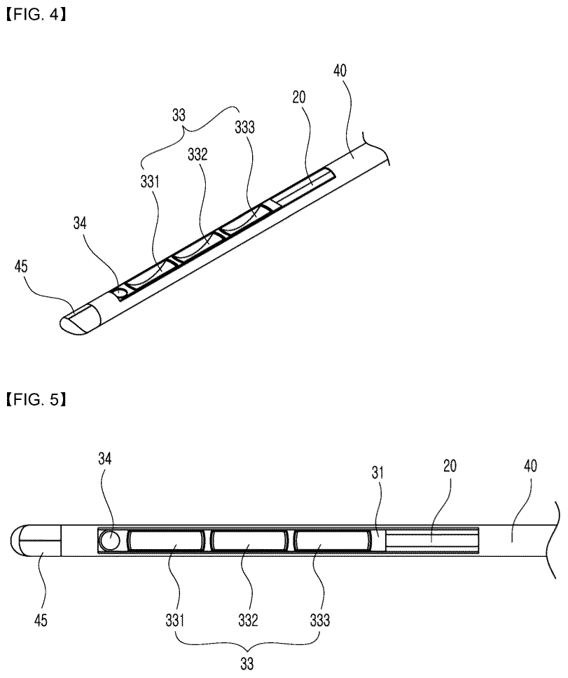

Using the image acquired by the imaging ultrasound transducer 34, a cross-sectional image of the target tissue, such as the prostate along the urethra, can be visualized. For example, as shown in FIG. 4, the imaging ultrasound transducer 34 may be implemented as a single-element piezoelectric device, i.e., a piezoelectric element for A-mode ultrasound imaging. An image can be obtained by moving the ultrasound transducer 34 in the longitudinal direction, and a cross-sectional image can be constructed based on the acquired data. As shown in FIGS. 4 and 5, to enable movement of the imaging ultrasound transducer 34, the transducer 34 may be installed on the housing 31 of the ultrasound probe 30, such that it linearly moves along with the movement of the ultrasound probe 30. In A-mode ultrasound imaging, the image acquisition speed varies depending on the physical movement speed of the single-element piezoelectric device. Therefore, the speed of linear movement during image acquisition may differ from the speed during focused ultrasound irradiation.

Based on the cross-sectional image acquired by the imaging ultrasound transducer 34, the position, shape, and size of the prostate can be identified, after which the ultrasound probe equipped with the ultrasound transducers 331, 332, and 333 can be moved to generate thermal coagulation points at the desired treatment site.

FIG. 6 is a diagram illustrating a procedure of performing an ultrasound treatment using the body cavity insertable ultrasound device according to an embodiment of the present invention. The ultrasound treatment apparatus according to an embodiment of the present invention may include the body cavity insertable ultrasound device and a display device for displaying the acquired ultrasound image. Before driving the ultrasound transducers 331, 332, and 333, the imaging ultrasound transducer 34 is moved across the region of interest to acquire an ultrasound cross-sectional image of the region. As shown in FIG. 6, the acquired ultrasound cross-sectional image may be displayed on the display device 111. The system may be configured such that the user can set a treatment region 113 on the display device 111. For example, the treatment region may be set using a mouse, or the display device 111 may be configured as a so-called touchscreen that can receive touch input from a human finger, touch pen, or the like.

As shown in FIG. 6, the positions of the focal points P1, P2, and P3 of the focused ultrasound beams U1, U2, and U3, corresponding to the positions of the ultrasound transducers 331, 332, and 33, can be displayed on the cross-sectional image where the treatment region 113 is indicated. In this state, where the treatment region 113 and the focal points P1, P2, and P3 of each ultrasound transducer 331, 332, and 333 are displayed on the display device 111, thermal lesions can be generated by ultrasound irradiation. By moving the ultrasound probe 30 without moving the sealing cover 40 and irradiating ultrasound, thermal lesions can be formed in a linear arrangement corresponding to the focal depths of each ultrasound transducer 331, 332, and 333. In this case, if a specific ultrasound transducer 331, 332, or 333 falls outside the designated treatment region 113, that transducer can be deactivated to prevent the formation of thermal lesions outside the treatment area due to focused ultrasound. By selectively driving the ultrasound transducers 331, 332, and 333 while moving them in consideration of the selected treatment region 113, thermal lesions can be formed only within the treatment region 113. The focused ultrasound beams U1, U2, and U3 displayed on the acquired cross-sectional image can be calculated based on the positions and geometric relationships between the imaging ultrasound transducer 34 and the focused ultrasound transducers 331, 332, and 333. Based on this, the positions of the focal points P1, P2, and P3 can be displayed in real time on the display device 111 as the ultrasound transducers 331, 332, and 333 move.

At this time, in order to optimize the ultrasound treatment, the plurality of ultrasound transducers 331, 332, and 333 may be arranged sequentially according to their focal depths. For example, as shown in FIG. 6, the ultrasound transducer U1 having the greatest focal length may be positioned at one end, and adjacent to it, the ultrasound transducers U2 and U3 may be sequentially arranged. By performing the ultrasound procedure while moving the ultrasound transducers 331, 332, and 333—arranged in this manner-into the treatment region starting from one end, it is possible to eliminate or minimize the need for reverse movement of the ultrasound probe 30. In addition, since it becomes difficult for focused ultrasound to create thermal coagulation points beyond already formed lesions, it is advantageous to position the ultrasound transducer 331 with the deepest focal point at the leading edge in the direction of movement. As illustrated in FIG. 6, multiple thermal lesions can be formed within the treatment region 113 by initiating the ultrasound transducers 331, 332, and 333 from either the left or right end of the treatment region 113 and repeatedly irradiating ultrasound while moving over a specified distance.

When the ultrasound transducers 331, 332, and 333 generate focused ultrasound while moving linearly, they may emit focused ultrasound sequentially one at a time, or multiple transducers may simultaneously emit focused ultrasound during the linear movement.

The linear movement of the ultrasound probe 30 and the operation of the ultrasound transducers 331, 332, and 333, as described above, may be controlled by the power/control device 100 mentioned earlier. To this end, the power/control device 100 may include a microprocessor, memory, and related hardware and software. For example, the microprocessor may be programmed to control the position of the ultrasound probe 30 and the operation of the ultrasound transducers 331, 332, and 333 as described above.

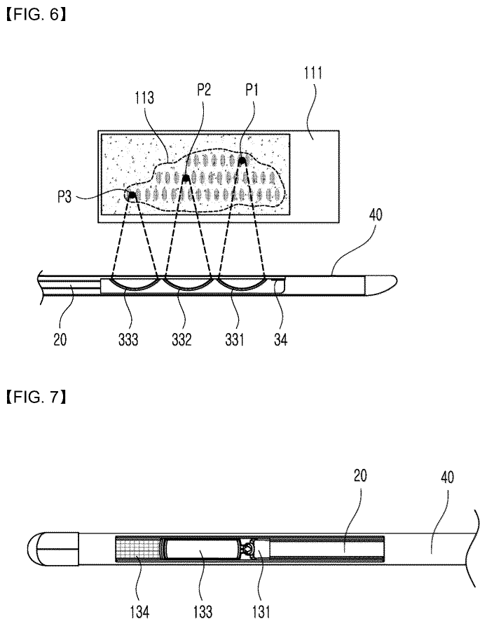

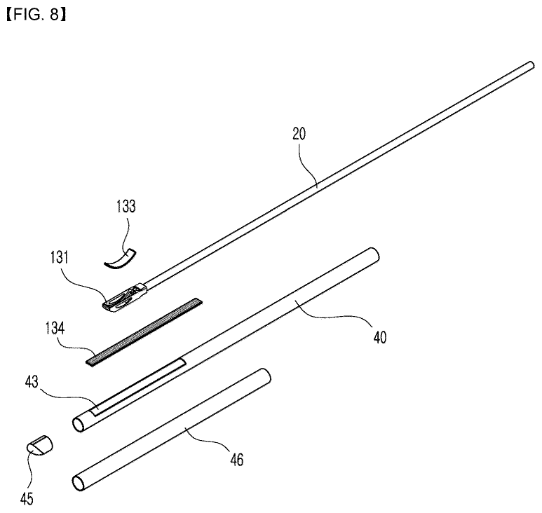

Referring to FIGS. 7 and 8, according to another embodiment of the present invention, the imaging ultrasound transducer 134 may be implemented as a multi-element piezoelectric device, i.e., a piezoelectric element for B-mode ultrasound imaging. The ultrasound transducer 134 may be installed on the sealing cover 40 independently of the movement of the ultrasound probe 30, since the imaging ultrasound transducer 134 capable of acquiring B-mode images can generate cross-sectional images without movement. As illustratively shown in FIG. 7, the imaging ultrasound transducer 134 may be positioned at the rear of the ultrasound probe 30, at a portion of the sealing cover 40 where the ultrasound transmission window 134 is formed. In another embodiment, the length of the ultrasound transducer 134 capable of B-mode imaging may be reduced, and it may be installed on the housing 31 of the ultrasound probe 30 so that it can move together with the probe.

FIGS. 10 and 11 illustrate a body cavity insertable ultrasound device according to another embodiment of the present invention, in which the ultrasound probe 30 is configured to be rotatable. In this embodiment, the ultrasound probe 30 is configured to allow not only linear movement, as described above, but also rotational movement. The same reference numerals are used for parts identical to those of the previously described embodiment, and redundant descriptions are omitted.

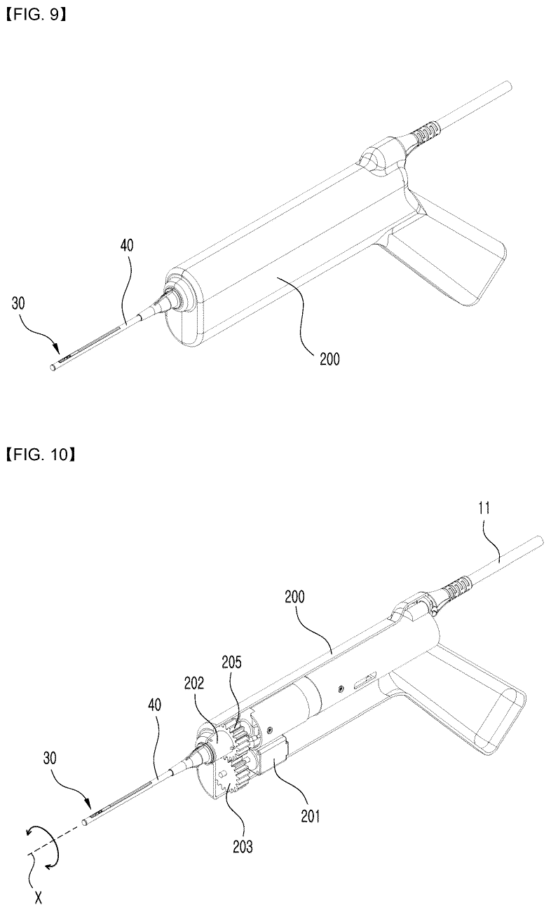

In this embodiment, the portion corresponding to the handle in the previously described embodiment is configured with a dual-structure design. Referring to FIGS. 9 and 10, a body 202 that supports the support bar 20 is provided, and the support bar 20 is supported by the body 202. At this time, the support bar 20 is configured to allow linear movement, as in the previous embodiment, to enable the linear movement of the ultrasound probe 30. A motor 61, an output shaft 62, and a rotating element 63 for the linear movement of the support bar 20 are housed within the body 202. The sealing cover 40 is fastened to the body 202.

The body 202 is rotatably disposed within the outer case 200 about a rotation axis X. In this configuration, the support bar 20 and the sealing cover 40 are connected to the body 202 and rotate about the rotation axis X when the body 202 rotates. The outer case 200 is configured to accommodate the body 202 and, as shown in FIG. 10, may have a shape that allows a user to hold it by hand, and it may also be fixed externally.

A motor 201 for generating rotational drive force to rotate the body 202 is disposed within the outer case 200, and a first gear 203, which rotates by the motor 201, is engaged with a second gear 205 provided on the body 202. As a result, when the motor 201 operates, the body 202 is rotated. As previously described, the rotation of the body 202 leads to the rotation of the support bar 20 and the sealing cover 40. The power/control device 100 described above may control the operation of the motor 201.

Various procedures can be performed using the ultrasound probe 30 capable of linear and rotational movement. For example, an image of the treatment region can be acquired through linear movement, and thermal coagulation points can be formed by generating focused ultrasound. After that, the ultrasound probe 30 can be rotated by a certain angle, and then linear movement can be performed again to acquire an image and generate focused ultrasound to form additional thermal coagulation points. The repeated execution of this process is useful for reducing tissues that surround the urethra, such as the prostate.

As another example, a rotational cross-sectional image of the treatment region may be acquired while rotating the ultrasound probe 30. After setting the treatment region based on the image, focused ultrasound may be irradiated while rotating the ultrasound probe 30 to form thermal coagulation points.

As another example, a cross-sectional image along the longitudinal direction of the ultrasound probe 30 may be acquired by performing linear movement across the entire range of the treatment region. Then, after rotating the probe by a certain angle, another linear scan can be performed to acquire additional cross-sectional images. By repeating this process, a three-dimensional ultrasound image of the treatment region can be constructed through software. Conversely, a 360-degree ultrasound cross-sectional image may be acquired by rotating the ultrasound probe 30, followed by linear movement over a certain distance, and then performing another 360-degree rotation to acquire additional cross-sectional images. By repeating this process, a three-dimensional ultrasound image of the treatment region can also be obtained via software. After marking the treatment area on the acquired 3D image and irradiating focused ultrasound accordingly, the entire treatment region can be treated at once, thereby reducing treatment time and improving user convenience. In this case, the focused ultrasound may be irradiated while performing linear movement, rotational movement, or a combination of both-first linear, then rotational.

While the embodiments of the present invention have been described above, the scope of the present invention is not limited thereto. It should be understood that various changes and modifications can be readily made by those skilled in the art without departing from the spirit and scope of the invention, and such modifications and equivalents are intended to be included within the scope of the present invention.

1. A body cavity insertable ultrasound device comprising:
a support bar extending in a longitudinal direction;
an ultrasound probe comprising one or more ultrasound transducers supported by the support bar and configured to be inserted into a body cavity and to emit focused ultrasound;
an imaging ultrasound transducer configured to acquire an image of tissue on which the focused ultrasound emitted from the ultrasound transducers acts; and
a sealing cover configured to surround at least a portion of the ultrasound probe and the support bar.

2. The body cavity insertable ultrasound device according to claim 1, wherein the ultrasound probe is configured to be capable of linear movement.

3. The body cavity insertable ultrasound device according to claim 2,
wherein the imaging ultrasound transducer comprises a piezoelectric element for A-mode ultrasound imaging, and
wherein the piezoelectric element for A-mode ultrasound imaging is installed on the ultrasound probe so as to move linearly together with the ultrasound probe.

4. The body cavity insertable ultrasound device according to claim 1, wherein the imaging ultrasound transducer comprises a piezoelectric element for B-mode ultrasound imaging.

5. The body cavity insertable ultrasound device according to claim 4, wherein the ultrasound probe is configured to be capable of linear movement, and
wherein the piezoelectric element for B-mode ultrasound imaging is configured to extend along the direction of the linear movement of the ultrasound probe.

6. The body cavity insertable ultrasound device according to claim 1, wherein the ultrasound probe comprises a plurality of ultrasound transducers having different focal depths, and
wherein the plurality of ultrasound transducers are arranged in order of their focal depths.

7. The body cavity insertable ultrasound device according to claim 6,
wherein the ultrasound probe is configured to be capable of linear movement,
wherein the ultrasound probe is configured to emit the focused ultrasound while passing through a target treatment region by linear movement, and
wherein the ultrasound probe is configured to move in a direction such that the ultrasound transducer having the greatest focal depth among the plurality of ultrasound transducers enters the treatment region first during the focused ultrasound irradiation.

8. The body cavity insertable ultrasound device according to claim 7, wherein each of the plurality of ultrasound transducers having different focal depths is individually controlled such that, when the focused ultrasound is emitted while passing through the treatment region, the focused ultrasound is not emitted if the focal point of the focused ultrasound falls outside the treatment region.

9. A body cavity insertable ultrasound device comprising:
an outer case;
a body rotatably disposed within the outer case about a predetermined rotation axis;
a support bar fastened to the body so as to be linearly movable relative to the body and rotatable together with the body about the rotation axis;
an ultrasound probe comprising one or more ultrasound transducers supported by the support bar and configured to be inserted into a body cavity and to emit focused ultrasound;
an imaging ultrasound transducer configured to acquire an image of tissue on which the focused ultrasound emitted from the ultrasound transducers acts; and
a sealing cover fastened to the body so as to rotate with the body about the rotation axis, the sealing cover being configured to surround at least a portion of the ultrasound probe and the support bar.

10. An ultrasound treatment apparatus comprising:
a support bar extending in a longitudinal direction;
an ultrasound probe comprising one or more ultrasound transducers supported by the support bar and configured to emit focused ultrasound while being insertable into a body cavity, the ultrasound probe being configured to be capable of linear movement;
an imaging ultrasound transducer configured to acquire an image of tissue on which the focused ultrasound emitted from the ultrasound transducers acts;
a sealing cover configured to surround at least a portion of the ultrasound probe and the support bar;
a display device configured to display the image acquired by the imaging ultrasound transducer; and
a power/control device configured to control the linear movement of the ultrasound probe and the operation of the one or more ultrasound transducers.

11. The ultrasound treatment apparatus according to claim 10, wherein the display device is configured to allow a treatment region to be indicated on the acquired image, and
wherein the power/control device is configured to control the movement of the ultrasound probe and the operation of the one or more ultrasound transducers in consideration of the treatment region.

12. The ultrasound treatment apparatus according to claim 11, wherein the one or more ultrasound transducers are configured to respectively generate focused ultrasound beams having different focal depths,
wherein the power/control device is configured to control linear movement of the ultrasound probe such that the ultrasound probe passes through the treatment region, and
wherein the power/control device is further configured to individually control the plurality of ultrasound transducers having different focal depths such that no focused ultrasound is emitted when the focal point of the focused ultrasound falls outside the treatment region as the ultrasound probe passes through the treatment region.

13. The ultrasound treatment apparatus according to claim 10, wherein the power/control device is configured to control the ultrasound probe to emit the focused ultrasound while passing through a target treatment region by linear movement, and
wherein the power/control device is configured to control the ultrasound probe to move in a direction in which the ultrasound transducer having the greatest focal depth among the plurality of ultrasound transducers enters the treatment region first.

14. The ultrasound treatment apparatus according to claim 10, wherein the imaging ultrasound transducer comprises a piezoelectric element for A-mode ultrasound imaging, and
wherein the piezoelectric element for A-mode ultrasound imaging is installed on the ultrasound probe so as to move linearly together with the ultrasound probe.

15. The ultrasound treatment apparatus according to claim 10, wherein the imaging ultrasound transducer includes a piezoelectric element for B-mode ultrasound imaging, and
wherein the piezoelectric element for B-mode ultrasound imaging is configured to extend along the direction of linear movement of the ultrasound probe.

16. The ultrasound treatment apparatus according to claim 10, wherein the ultrasound probe is configured to be rotatable about a predetermined rotation axis in addition to being capable of linear movement.
